(19) United States
(12) Patent Application Publication
(10) Pub. No.: US 2012/0078328 A1
(43) Pub. Date: Mar. 29, 2012

(54) SYSTEM AND APPARATUS FOR TREATMENT OF BIOLOGICAL CELLULAR STRUCTURE WITH ELECTROMAGNETIC WAVE ENERGY AND ELECTROMAGNETIC FIELD ENERGY SOURCES

(76) Inventors: Marc Vancraeyenest, Ooskamp (BE); Philippe Vandemoortele, Knokke (BE); Covington Stanwick, Annapolis, MD (US)

(21) Appl. No.: 12/891,092

(22) Filed: Sep. 27, 2010

(51) Int. Cl. A61N 5/06

(52) U.S. Cl. 607/88

(57) ABSTRACT

A system and apparatus for treatment of cellular structure anomalies, principally in human patients, utilizes precisely controlled electromagnetic wave energy and electromagnetic field energy. The former energy is generated from a combination of coherent and incoherent light energy which is joined and focused on patients thru a unique arrangement of faceted coated reflectors and filters. The latter energy is generated in part by unique coils surrounding the light sources which include coils wound with bifilar wire. The disclosed apparatus also includes a platform, preferably segmented, that facilitates alignment of a patient's bodily area needing treatment. Additional coils are placed in the platform in strategic locations proximate the area of the patient to be treated. A digital control module can be programmed to regulate the frequency, duration, phase, duty cycle, pulse and amplitude of all energy sources in the claimed systems.

### BACKGROUND OF THE INVENTION

This invention relates to a system and apparatus for influencing biological cellular structure using electromagnetic wave energy, principally in the visible and near infrared light spectrum, and electromagnetic field energy.

This invention relates, in particular, to a system for realising beneficial effects on a biological cellular structure in a human or animal. More particularly, it relates to a system for achieving specific therapeutic effects in humans and animals.

Electromagnetic wave energy of certain frequencies can create certain therapeutic effects on a living creature. Applications of this nature are generally referred to as “light therapy.” There are instruments for application of light therapy that comprise a so-called coherent light source, such as a laser system. These systems have the disadvantage that their use can be hazardous. Another example of light therapy is that used for treatment of neonatal jaundice, in which newborns are exposed to light emitting electromagnetic wave energy in the 420 to 470 nanometer wave length range. Electromagnetic wave energy in the range of 405-420 nanometers has been used to treat acne.

An instrument using light therapy is described in French Patent Application No. 2639834. With this instrument, light of a certain spectrum (about 500 nanometers) is used. This instrument is handheld and allows orienting the emitted light directly at affected tissue. This kind of instrument has limited application and is not very effective.

In addition to light therapy, electromagnetic field energy is used to obtain a beneficial influence on the health of a human. For example, pulsed electromagnetic field therapy (PEMF) is an FDA approved therapy to repair bone non-unions.

In Canadian Patent No. 1,301,255 an instrument is described which combines infrared light energy (900 nanometers) with electromagnetic field energy to treat human and animal organisms.

U.S. Pat. No. 7,744,522, issued to one of the inventors of this application, discloses apparatus for treating cellular structure with a combination of electromagnetic wave energy and electromagnetic field energy. The apparatus and systems disclosed herein provide a substantial improvement over those disclosed in this earlier patent.

As used herein, the term “electromagnetic wave energy” will be generally used to describe specific wave lengths of visible light and near IR spectrums and “electromagnetic field energy” (EMF) to describe energy fields emanating from electromagnetic coils. The former refers to electromagnetic energy in wave form ranging nominally from 380 to 880 nanometers. The term electromagnetic field energy (EMF) as used herein refers to electromagnetic fields in the general range of less than 0.1 mT (milli-Telsa) at 30 cm to about 10 mT at 0 cm.

### SUMMARY OF THE INVENTION

The National Institutes of Health (NIH) through its National Center for Complementary and Alternative Medicine (NCCAM) has recognized the efficacy of the application of measurable energy forces and fields to diagnose and treat medical conditions. NCCAM generally refers to these approaches as “Veritable Energy Medicine” (VEM). A well established VEM therapy is the neoBLUE® LED Phototherapy system for treatment of newborn jaundice. This VEM therapy meets American Academy of Pediatrics clinical practice guideline: Management of hyperbilirubinemia in the newborn infant. Also the FDA approved NeuroStar TMS Therapy System (www.neuronetics.com/prod-system.aspx) uses VEM magnetic fields.

The object of this invention is to provide a comprehensive system and apparatus which substantially improves and optimizes the therapeutic effect of veritable (measurable) energies of light and electromagnetic fields applied to the entire human body.

This objective is achieved in the claimed system and apparatus using vastly improved sources of light energy and electromagnetic field energy, plus ultra-precise digital control means to adjust and modulate the frequency, phase, duration, duty cycle, pulse and the amplitude of the light and electromagnetic fields so that specific resonances and interferences may be obtained in the human biological cellular structure.

It has been found that therapeutic treatments based on EMF alone, or in combination with electromagnetic wave energy can be improved by enhancing the structure, placement and control of electromagnetic coils generating EMF. Coils comprising multiple windings of specialized wire composition have been found by applicants to be particularly effective in these treatments.

The effects of electromagnetic wave energy and EMF on a living organism can be explained as follows. Each living organism is an electromagnetic field that, precisely because of its physical properties, is a living creature. All parts, for instance, of the human body collectively comprise one-hundred trillion cells. Each cell is built from complex compounds that themselves are made up of molecules. Each molecule is made up of atoms and each atom has its own electromagnetic field and wave energy force created by the relations of positively charged protons surrounded by negatively charged electrons. The homeostatic function of this phenomenon is essential for all human life.

Hormones, vitamins, (trace) elements, lipids, amino acids, carbohydrates, salts, bases, acids, but also water, oxygen, carbonic acid gas and nitrogen have, by their molecular composition, a proper chemical identity from the periodic system of elements. In addition, it is known that the DNA of the cells, and also water molecules, in a living organism, a human being one example, are polarized. All of these have a specific electromagnetic field or charge, which is built up and composed of the corresponding atoms. One could state that the sum of the electrical charge of each atom in a molecule gives such molecule a specific vibration. Thus, the sum of the molecules which make up a cell give that cell a specific vibration. This is referred to herein as the “specific vibration frequency” or “resonance frequency” of cells. Consequently, cells, tissues and organs also have electromagnetic properties.

It is a well established fact that atoms in a cell are capable of being energized by external electromagnetic spectrum energy forces, particularly, electromagnetic wave energy and electromagnetic field energy (EMF). These external electromagnetic energy forces are capable of triggering biological electro-chemical reactions in cellular structures. The system and apparatus described below provides substantial flexibility and efficacy in that regard. Through experimentation it has been concluded that improved treatment of cellular structure can be obtained when delivered energy frequencies lie within a rather narrow range.

By creating resonances, electrical currents, action potentials and bio-electrical communications in specific cells, thereby triggering biochemical reactions, it has been found that it is possible to influence biological cellular structures in order to obtain a specific therapeutic effect.

Achieving this improved influence on the cellular structure of humans and animals is accomplished by providing an integrated system to direct electromagnetic wave energy (light therapy) and electromagnetic field energy (EMF) upon cellular structures of the body. Harnessing appropriate electromagnetic wave spectrum output capabilities and electromagnetic field energy potential generation into a compact, versatile system has enabled breakthrough cellular reformation.

An important element in the applicants' system and apparatus for administration of these forces to control cellular behavior is a Photon-Magnetic Energy Source (PMS). In a preferred embodiment of the PMS, a dome-like structure contains coherent and incoherent light sources, specially coated and uniquely faceted parabolic glass and polycarbonate reflectors, polarization lens filters, a Tri-Wind Magnetic Coil Assembly and control electronics. The incoherent light source preferably emits light in nominal wave lengths of 380-840 nanometers and resides at or near the top of the PMS. It is surrounded by a faceted, coated parabolic reflector which permits transmission of certain wave lengths and reflects, bundles and precisely projects the remaining wavelengths of incoherent light toward a patient in a controlled and precisely specified beam angle/field culminating in a homogeneous spot. Between this light source and the patient are one or more polarization filters. The ultimate result is a spot with substantially no thermal effect (a so-called cold light source) nor ionizing radiation where the spot impinges on the cellular structure being treated.

Coherent light emanating from the PMS is provided by multiple, preferably eighteen (18), sources of coherent light such as light emitting diodes (LEDs). The LEDs preferably form a circle under the base of the parabolic reflector. Each source of coherent light is, in turn, surrounded by its own parabolic faceted reflector which bundles and focuses the coherent light in a defined beam on the person being treated. Between this light source and the patient are one or more polarization filters which further improves the selectivity of the electromagnetic wave energy reaching the patient.

Around the lower edge of the PMS is a unique coil configuration capable of emitting electromagnetic field energy under very precise control of frequency, phase, duration, duty cycle, pulse and amplitude. Preferably, three distinct coils are arranged in a holder at the lower circumference of the PMS, each of which can be separately controlled as to frequency, phase, duration, duty cycle, pulse and amplitude of the EMF being produced. One of these coils is wound from a special bifilar wire alloy and coating that substantially enhances its performance characteristics.

The PMS is part of an overall therapeutic treatment system and apparatus including a segmented structure which supports a patient during treatment which is referred to herein as a Human Platform (HP). The Human Platform can be oriented from a seated to fully reclining position. The Human Platform is supported by an Electromechanical-Electronics Cabinet (EEC) or base which, as the name suggests, houses mechanisms for controlling some of the components of the overall system. The EEC also contains some electronics which regulate the devices emitting electromagnetic wave energy and electromagnetic field energy.

The Human Platform preferably contains additional electromagnetic coils emitting EMF. These coils are strategically located and imbedded in the Human Platform in proximity to where specific bodily regions of the human undergoing treatment will be positioned. Each coil is capable of being controlled separately or in any combination or sequence with the other coils to most effectively treat the patient.

At the heart of the system is a digital electronic control module (CM) strategically located adjacent the Human Platform which precisely controls each function of the PMS, the HP and related systems.

The system in accordance with this invention is preferably configured to generate a therapeutic effect in a human or animal body.

### DETAILED DESCRIPTION OF THE DRAWINGS

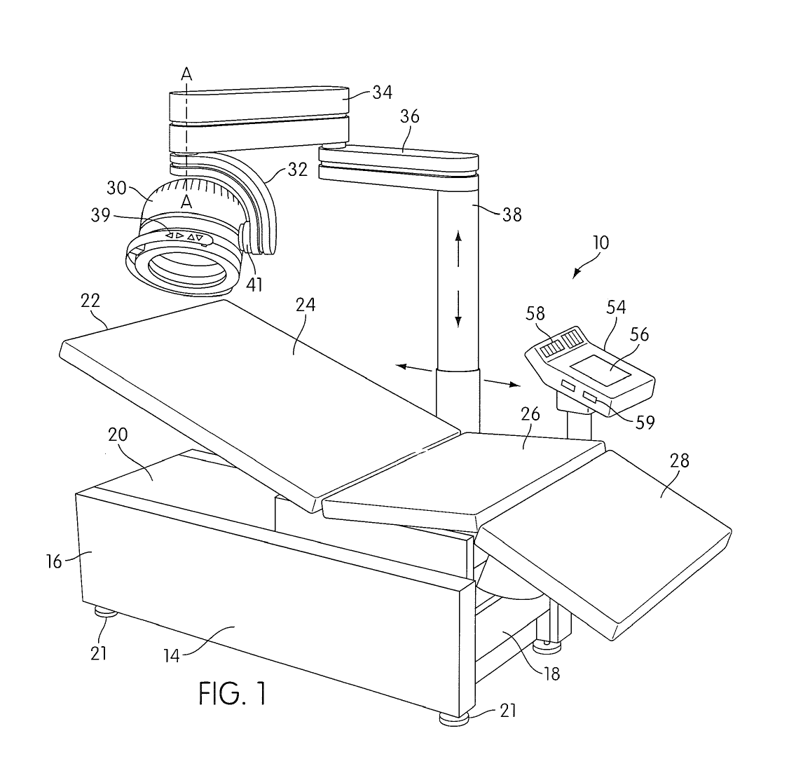

FIG. 1 is perspective view of the several apparati which comprise the inventors' system for treatment of biological cellular structure including the Photo-Magnetic Energy Source (PMS), Human Platform and Control Module;

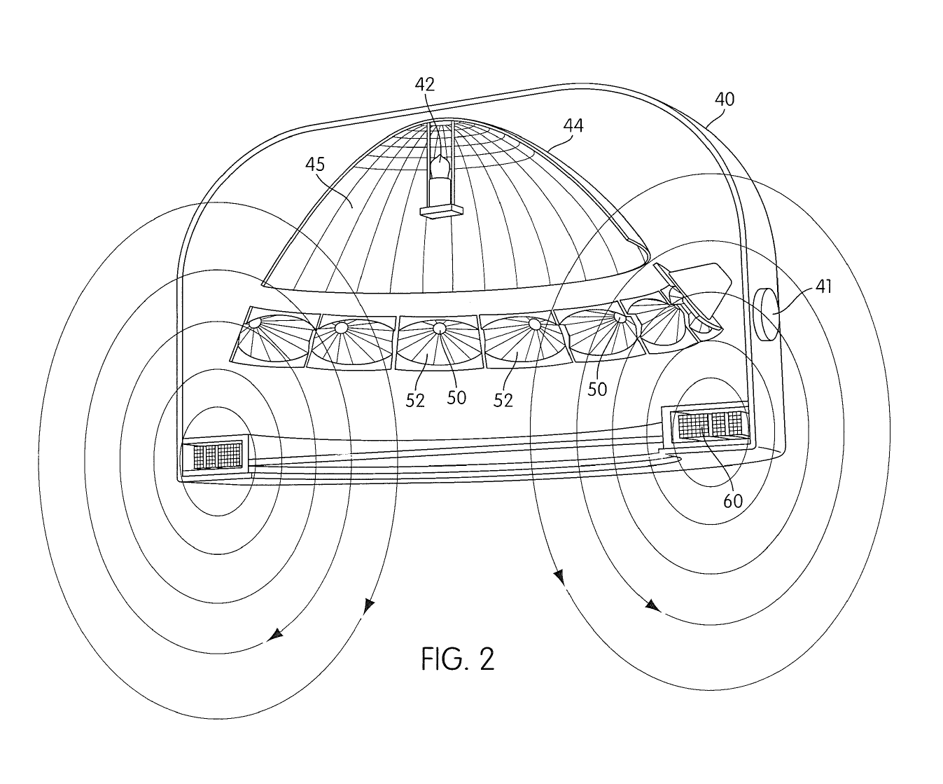

FIG. 2 is a view in partial cross-section of the PMS illustrating placement of the incoherent light source, parabolic reflector, polarization lens, coherent light sources and reflectors plus specially wound EMF coils;

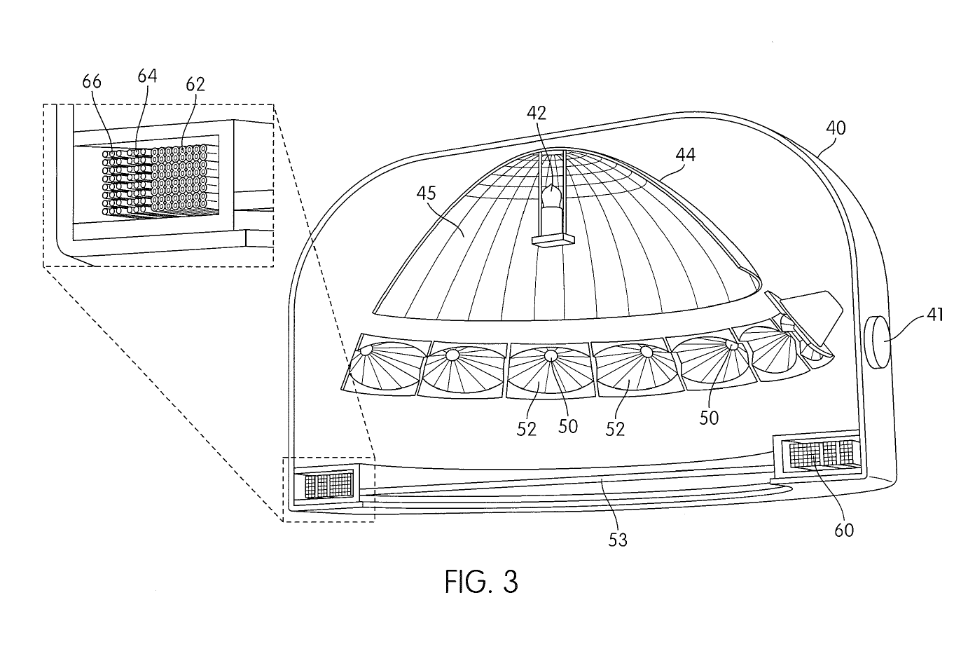

FIG. 3 is a partial cross-sectional view of the PMS with an exploded view of the EMF coils;

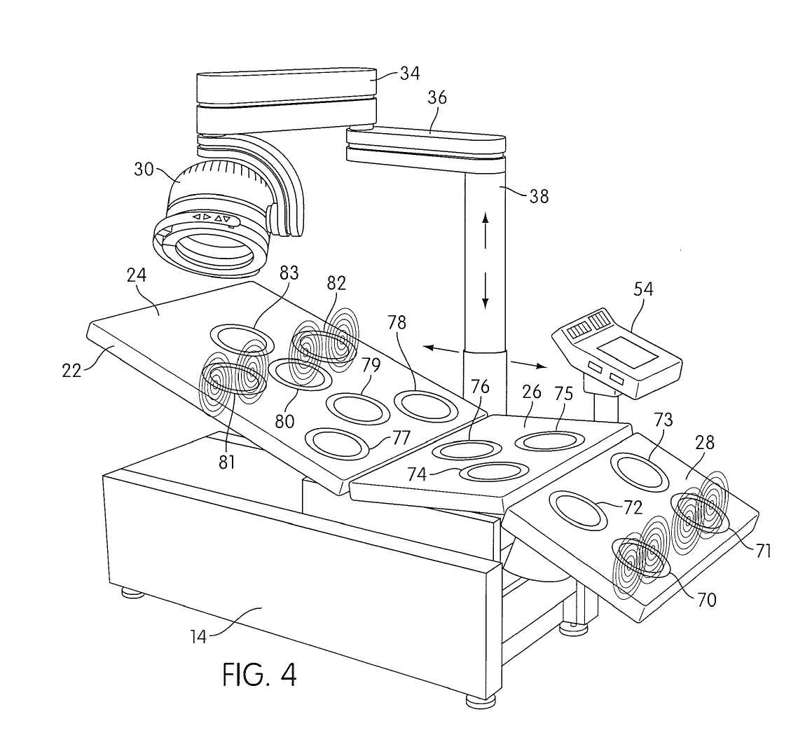

FIG. 4 is a pictorial view of the Human Platform illustrating a preferred placement of electromagnetic coils in strategic areas of that Platform;

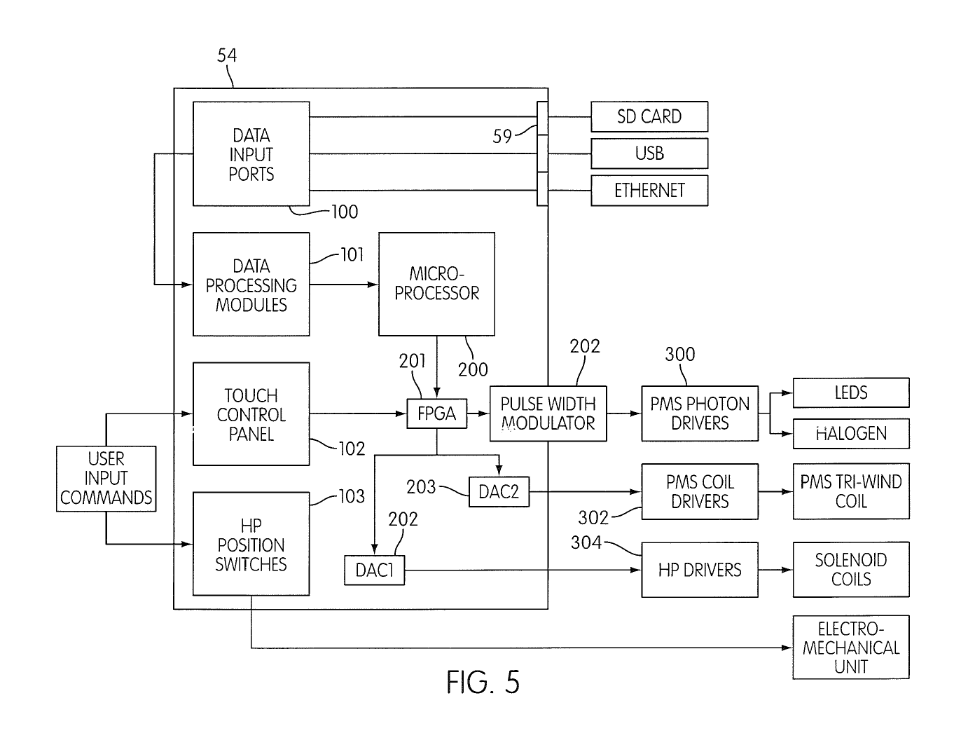

FIG. 5 is a block diagram of some components of a Control Module used to control the system and apparatus of this invention;

### DETAILED DESCRIPTION OF THE INVENTION

The overall arrangement of apparatus used in this system 10 for treatment of cellular structures is illustrated in FIG. 1. This apparatus includes a cabinet 14 which forms a structural base for the disclosed components of the disclosed system. The cabinet 14 has vertical walls 16, 18 and a partial top 20 that provide the structural integrity and enclosure for cabinet 14. Suitable leveling feet 21, or wheels, at the corner of cabinet 14 may be used to facilitate portability and/or insure stability of the overall apparatus of this invention. Within the cabinet 14 are suitable electromechanical mechanisms and electronics (not shown) to facilitate placement of a patient being treated. Also within cabinet 14 are essential electrical and electronic components such as power supplies, switches, controls, drivers, etc. (not shown) which facilitate operation of the electromagnetic wave energy and electromagnetic field energy sources of the disclosed apparatus.

A Human Platform (HP) 22 is arrayed above, and connected to, the cabinet 14 and is preferably segmented so that it can be fully adjusted to place a person in the optimal desired position for treatment. As illustrated in FIG. 1, the Human Platform is divided into three segments: a head, upper body segment 24, a hip/thigh segment 26 and a leg/foot segment 28. Each segment of the Human Platform can be adjusted to the most beneficial position for treatment utilizing conventional electro-mechanical actuators in the EEC cabinet 14.

The Photon-Magnetic Energy Source (PMS) 30 is capable of being arrayed at any angle and elevation relative to the Human Platform 22. This is accomplished by rotatably mounting the PMS to an arcuate arm 32 at bearing 41. Arcuate arm 32 is rotatable about an axis A-A in a Swivel Arm (SA) 34 (see FIG. 1). The Swivel Arm 34 may be rotated relative to another Swivel Arm 36 to further improve the ease of placing the PMS in a position most advantageous for treatment of patients.

The Swivel Arms 34 and 36 are further rotatably mounted atop a Swivel Arm Mast (SAM) 38. The lower end of the mast 38 is preferably attached to the EEC cabinet with rails, slots or other means (not shown) so that it may be moved along one side of EEC cabinet 14 from a position proximate the patient's torso or legs toward the patient's head or vice-versa. The Swivel Arm Mast 38 is preferably also moveable in a vertical direction to again foster placement of the PMS in the most advantageous location proximate the patient being treated. Adjustment of the PMS 30 relative to the Swivel Arm Mast 38 is preferably done manually. Movement of the Swivel Arm Mast along the EEC cabinet base 14 or vertically relative to the Human Platform 22 is preferably done using electro-mechanical means within the EEC base cabinet. This movement is controlled by control buttons 39 on the PMS.

The PMS 30 includes an outer casing 40 with a bearing 41 for rotatable attachment of the PMS to the arcuate arm 32. Within the upper portion of casing 40 of the PMS is a source of incoherent light 42 as shown in FIG. 2. This source of incoherent light 42 is preferably a 250 watt halogen bulb, although any source or wattage that emanates electromagnetic wave energy in the nominal range of 380-840 nanometers can be used. This light 42 is mounted in a position on the center line and in the top third portion of reflector 44 as illustrated in FIG. 2. The incoherent light source 42 is surrounded by a parabolic glass reflector 44 whose unique coated internal faceting 45 facilitates direction of the incoherent light towards the patient in a focused beam resulting in a defined spot. The interior surface of the faceted parabolic reflector 44 preferably receives different thin metal-oxide coating layers, preferably of quartz and/or titanium, which perform the following functions. These coating layers allow for the reflection of most, but not all, of the incoherent light wavelengths emanating from the incoherent light source 42. These coatings selectively allow transmission of certain unwanted wave lengths of the electromagnetic wave energy emitted by the incoherent light source 42 while downwardly reflecting and collimating electromagnetic wave energy through the opening in the reflector 44 towards a patient. It has been found that incoherent light is most effective in therapeutic treatment if it is within a preferred wavelength range of 400-780 nanometers and is focused on the patient in a homogenous spot about 7-10 inches in diameter. This focusing of light with homogeneous energy inputs across the area of the spot is enabled by use of the coated parabolic faceted reflector 44 disclosed herein.

Multiple sources of coherent light 50 are arranged around the inner perimeter of the PMS casing 40 (see FIG. 2). Preferred coherent light sources 50 are light emitting diodes (LEDs) in the 690-880 nanometer wavelength range. Preferably LEDs of varying wavelengths are used. This facilitates greater potential control of the electromagnetic wave energy emitted by the LEDs. For example, in a PMS containing eighteen (18) LEDs, one-third of the LEDs could have a nominal wavelength of 690 nanometers and the rest a nominal wavelength of 870 nanometers. The LEDs can be adjusted in number and nominal wavelength to suit the situation. Each of the coherent light sources 50 is preferably surrounded by a parabolic, faceted reflector 52 which directs its coherent light toward the patient in a focused beam resulting in a defined spot which overlaps and contributes to the homogeneity of the spot where incoherent light strikes the patient and is preferably within the same 7-10 inch diameter spot cast by incoherent light. The reflector 52 preferably comprises a molded poly carbonate material with a silver metallic reflective surface. A polarization filter 53 extends across the exit area of the PMS to further control the electromagnetic wave energy emanating therefrom. (See FIG. 3.)

The output and frequency of the coherent and incoherent light sources is regulated by the Control Module 54. The Control Module contains an LCD touch screen display panel 56 for manual entry of data and operating instructions. The Control Module is preferably mounted on, or adjacent, the cabinet 14 in an area that is readily accessible to the operator of the claimed system 10 (See FIG. 1).

The periphery of the lower (as viewed in FIG. 2) open end of PMS casing 40 contains a Tri-Wind Magnetic Coil Assembly 60 which is capable of generating unique electromagnetic field energy (EMF) due to its composition and control by the Control Module 54. As the name suggests, the Tri-Wind Coil has three components. Those components are described below with reference to FIG. 3 which includes an enlarged cross-sectional view of the Tri-Wind Coil 60.

That coil comprises three seperate windings as illustrated in FIGS. 2-3. The improved coils are, in part, based on use of special alloys and specially shaped wires matched with appropriate coatings for each winding. Of the three separate windings each plays a role in influencing penetration of photons generated by the coherent and incoherent light sources into the person being treated. The Tri-Wind Coil 60 is also a powerful source of electromagnetic field energy useable for patient treatment.

One of the windings 62 in the Tri-Wind Coil 60 is preferably made from a unique alloy and wire configuration. This winding is formed from a bifilar wire which is preferably made from a stainless steel alloy of high permeability and resistance. Preferably the bifilar wiring used in the first coil has a dog bone cross section comprising two stainless steel wire strands embedded in a coating of polyimide with each wire electrically isolated from the other. For best performance this coating should be from 20-33 microns, nominal, for each wire.

The bifilar wire is continuously wound into position around a circular coil holder (spool) to form the first coil 62. Positive and negative leads from each conductor of the bifilar wire are connected to the Control Module 54.

The second winding 64 in the Tri-Wind Coil preferably comprises coated copper wire with appropriate leads to the Control Module 54. This winding is preferably wound on the outside of the first winding 62 containing the bifilar wire. In one embodiment the composition of the coating around the wire is such that it will adhere to adjoining layers of copper wire coating as the winding is formed.

A third winding 66 can be used to enhance the EMF generated in the Tri-Wind Coil. It, like the second winding, is preferably formed from coated copper wiring. Both the second and third winding have appropriate leads, separate from each other, which are connected to the Control Module 54.

Intermediate the Tri-Wind Coil 60 and the Control Module 54 are preferably multiple drivers which control signal inputs for each of the specified windings in the coil. The drivers can vary the electrical impulse to each winding in a variety of ways, including the duration and wave form (square, sinus, saw tooth, wave, etc.) of the electrical impulse. This permits an infinite variety of electrical inputs and consequent variety of electromagnetic fields that can be created according to the desired treatment regimen via the Control Module inputs. Thus, the drivers must be capable of operating in the range of microseconds as instructed by inputs from the Control Module 54 to modulate the frequency, duration, phase, duty cycle, pulse and amplitude of the electrical impulse sent to each of the coil windings. The windings thereby are capable of generating the electromagnetic impulses in a fashion which supplements the acceleration of the photons generated by the light source. This enables control over that combination in treatment of varying parts of the body as discussed above, by which tissue treatment at varying depths within the body can be realized.

The Control Module 54 also controls the light sources 42 and 50. It can adjust the frequency, phase, duration, duty cycle, pulse and amplitude of electromagnetic wave energy forces emitted from these sources to provide effective treatment of biological cellular structure. For example, the Control Module can selectively activate only those LEDs emitting wave lengths of 690 nanometers from among the numerous LEDs in the PMS. Or the Control Module can selectively activate LEDs 50 of one or more other wavelengths from among the many LEDs arrayed around the inner periphery of PMS 30. In addition, the Control Module can control the instantaneous frequency, phase, duration, duty cycle, pulse and amplitude of the selected LEDs.

The Human Platform 22 can be modified to incorporate additional coils 70-83 to complement the electromagnetic energy fields emanating from Tri-Wind Coil 60. These platform coils 70-83 as, for example, illustrated in FIG. 4 are precisely arranged in order to provide preferred coverage to the affected areas of the person being treated. The placement of the coils in the Human Platform 22 can be varied to accommodate different treatment regimens. A preferred arrangement shown in FIG. 4, illustrates coils 70-71 placed in the approximate location of a patient's lower extremities (ankles/feet) in segment 28 of Human Platform 22. Coils 72-75 are placed in segments 26 and 28 of the platform 22 in the approximate location of a patient's knee area. Treatment of an anomaly in the area of a patients sacrum is facilitated by the placement of coil 76 in segment 26 of Human Platform. In the upper body segment 24 of the Human Platform 22 are located coils 77-78 which are generally oriented to treat anomalies in the lumbar region of a patient's spine. Coils 79-80 are used to treat anomalies in the thoracic region of a patient. Coils 81-82 can be energized to treat anomalies in the cervical region of a patient and coil 83 can be energized to treat cranial anomalies. A typical diameter of the illustrated coils 70-83 is approximately 8 inches. Each is wound in coated copper wiring and each of the coils is separately controlled by the Control Module 54 via designated drivers in a manner similar to that described above with respect to the Tri-Wind Coil 60. The above described placement of coils in the Human Platform 22 can be varied depending on the focus of treatment desired.

The Control Module 54 includes a LCD touch screen 56 and manual actuation buttons 58 as illustrated in FIG. 1. The LCD is a graphical user interface used by a technician or doctor to deliver manual commands to the system and monitor the system's status while treatment is in progress. Manual actuation buttons 58 are principally used to adjust the height and angulation of the Human Platform 22 and its segments 24-28.

The Control Module is the input means for entering therapeutic programs for use in the disclosed system. In a preferred embodiment the Control Module 54 contains input ports 59 (See FIG. 1) on one surface thereof for inputting therapy programs unique to specific injuries and ailments to be treated.

FIG. 5 is a schematic diagram of the overall control functions of the disclosed system as driven by the Control Module 54. It is to be understood, however, that some of the functional components illustrated in FIG. 5 may be located in the cabinet 14 and the PMS 30 and electrically linked to the Control Module 54 by suitable wiring. As illustrated in FIG. 5, various input devices, including data input ports 100, are designed to receive the preprogrammed instructions for each known therapy to be practiced by the disclosed system. These input means include, for example, an SD card and computer based input. These inputs are communicated to the Control Module through ports 59 (see FIGS. 1 and 5) suitably sized to receive SD cards, USB cabling, Ethernet cabling, or the like.

This direct input of operating data reduces the amount of data needed to be stored in the Control Module. Also, by preselecting operating instructions from an external source, less time is needed to set up and start therapies, allowing for a greater utilization of the therapeutic benefits of the disclosed system and apparatus. Thus, for example, the operator of the system need merely select an SD card for a particular therapy to be practiced on a patient, insert it into slot 59 of the Control Module and immediately start therapy. The data is then processed in a microprocessor 200 which reads the inputted therapy protocol and transfers it to an FPGA circuit 201 for further processing. After processing the data, therapy instructions are stored in a SDRAM memory for later use.

The instructions inputted to the Control Module 54 are then distributed to the various components of the disclosed system, namely, the coherent and incoherent lighting, the PMS coils and Human Platform coils on pathways generally illustrated in FIG. 5. As illustrated in FIG. 5, the multiple sets of LEDs are controlled separately or individually, if needed, to provide the desired electromagnetic wavelengths for treatment of various conditions. A separate control of the incoherent Halogen light 42 also emanates from this source. Additional controls are directed to each of the three components of the Tri-Wind coil in the PMS. Lastly, separate signals are sent to coils in the Human Platform which enables control of these coils. Each of those multiple therapy channels can in turn be controlled by signals which create different waveforms, e.g., square, sinus, tooth, wave, etc. for the coils as well as multiple high speed modulated signals for the LEDs and one command signal for the Halogen lamp.

As illustrated in FIG. 5 the data inputs into data input ports 100 include SD card, USB and Ethernet data. The inputted data is further processed in data processing module 101. This processed data is then electrically communicated to microprocessor 200. After further processing in the microprocessor 200 the data is converted in module 201, the Field-Programmable Gate Array (FPGA). The FPGA contains a Pulse Width Modulator (PWM) 202 and Digital to Analog Conversion unit (DAC). This facilitates subsequent control of the various operating components including the LEDs 50, incoherent light 42, Tri-Wind Coils 60 and coils 70-83 imbedded in the Human Platform 22. The Pulse Width Modulator (PWM) 202 provides three high speed modulated command signals for the multiple groups of LEDs and another command signal for the Halogen lamp as illustrated in FIG. 5. The signal from the PWM is processed through appropriate drivers 300 to operate these light sources. The sequence of control elements permits the generation of operating signals with a time resolution of one microsecond or less allowing for careful control of the electromagnetic wave therapy emanating from the resolution from the lights in the PMS.

Digital analog converters 202 and 203 process the signals from the FPGA that are used to control the electromagnetic field energy emanating from the Tri-Wind Coil 60 and coils 70-78 in the Human Platform 22. The analog signals from DAC 1 are used to control drivers which are electrically connected with the coils in the Human Platform. These drivers are capable of varying the electrical impulse in each winding of the coils to regulate the duration in wave form of the electrical impulse delivered to the coils every microsecond. Similarly DAC 2 delivers signals to the Tri-Wind Coil through appropriate drivers as shown in FIG. 5.

The Human Platform position switches 103 are manually controlled by the operator through input 103 since those inputs generally have to be adjusted for the body shape, weight, height and mobility for each patient being treated. This is achieved through mechanical switches 58 preferably located in Control Module 54. These input switches 58 are directly linked to electromechanical units in cabinet 14 to raise and lower each segment 24-28 of Human Platform 22.

The Control Module 54 can be programmed to automatically generate and run through a pattern of specific frequencies, duration, phase, duty cycle, pulse and amplitude and thus, by means of precision settings, generate resonances in the cellular structure of tissues and bio-cellular matrix, in general in a biological cellular structure.

The two different light sources can be driven separately and combined in accordance with a specific modulation pattern and thus synchronously with the employed magnetic fields, this in order to modify, for instance, the reflection and absorption behavior of the treated tissue at a specifically to be set resonance frequency, by which very deep tissue penetration can be obtained.

Examples of the beneficial affects of this disclosed system are set forth below.

### Example 1

### Post Operative Wound Healing Intervention

A double Hallux Valgus operation was performed by an Orthopedic Surgeon on a women patient of age 52. The surgeries were performed on two consecutive days in an accredited Belgium Medical Institution by a renowned Orthopedic Specialist.

Normal patient recovery time for an operation of this nature is between 4 and 6 months as measured by full walking mobility. Additional metrics during a patient recovery period such as pain management/medication dosing, inflammation, wound closure rates, scarring, bone growth and implanted device rejection are also typically measured.

The patient began treatments with the system described herein utilizing its unique energy force emissions of electromagnetic waves and electromagnetic fields 24 hours after the last surgery was performed. A total of 19 treatments were given to the patient over the course of the 21 days post operation. These treatments ranged in duration from 47 minutes to 19 minutes. There were 5 treatment program protocol inputs into the system's Control Module during the course of treatment; specifically wound infection/inflammation, pain, wound closure, bone growth, scar tissue mitigation.

A summary of metric indicators are presented for comparison: (post operative):
Inflammation (foot swelling): swelling and wound inflammation went from severe on Day 1 of treatment to a 75% reduction by end of Day 3 treatment. A 100% reduction was achieved by Day 5. This is in contrast to conventional recovery inflammation reduction which may take several weeks or longer.
Wound infection: There were common signs of the presence of wound infection upon treatment start up. These signs diminished daily resulting in no signs of any wound infections at Day 5, as measured by registered nurses who changed surgical dressings daily. Infections are common in these cases and require topical and oral antibiotic administration only subsiding after 10 days or more.
Pain Management/Dosing: Pain medications were taken by patient every 2 hours at the start of treatment. This was reduced to 3 times per day by Day 3 of treatment and 1 per day by Day 5 and no medication by Day 7. Normal pain dosing continues for weeks and in heavy doses.
Bone Growth: X-Ray films on Day 7 indicated 75-80% bone union and no sign of device implant rejection. Normal bone union at these percentages occurs typically after several months.
Wound closure/scarring: Post operative wound closure was 30%. After Day 6 wound closure had reached 100% as verified by wound dressing nurse. Scarring and tissue discoloration were minimal by Day 13 and only slightly perceptible by Day 19. Typical full closure may take many weeks and complete scarring mitigation may not occur at all.
Full mobility: The ultimate metric is the patient's ability and time frame to gain partial and full walking mobility. The patient undergoing therapy was able to walk 50% of her normal walking activity utilizing special pressure shoes at Day 5; 100% by Day 8 still in the pressure shoes and at Day 21, after the follow up visit to the attending surgeon, patient left the office in normal shoes and resumed normal walking activities. Complete walking recovery takes normally takes 4 to 6 months.

### Example 2

### Post Operative Wound Healing and Hair Stem Cell Intervention

A 22 year old male underwent a Hair Stem Cell Transplantation procedure at a world renowned Clinic in Holland. The procedure involves the surgical extraction of numerous hair follicle tissues from a designated donor site on the patients head. These extracted tissues are then processed and the stem cells are harvested. Once this is accomplished the harvested stem cells are surgically transplanted by special technique and device to the designated transplantation site on the patients head.

In this particular procedure there were 1,715 stem cell implantations on the patients' head and a like number of wounds created at the stem cell donor site.

The day after the procedure was conducted the patient underwent a series of treatments with the disclosed system and apparatus utilizing its electromagnetic wave energy and electromagnetic field energy forces. The treatments lasted for a period of 30 days with the dosing daily for the first week and reduced to several times per week thereafter. Dosing durations were approximately 30-40 minutes.

The wound healing of both the patient donor site and the transplantation site was 50% complete by Day 5 of the treatment. Total skin wound healing (few random scabbed areas and normal patient skin tone, blood flow and nerve feeling) was achieved by Day 17.

In addition the re-growth of donor site hair follicles was present by Day 5. Likewise evidence of hair growth in each of the transplant sites was produced by Day 7 and increased weekly until Day 30 when hair length was ˜1 inch.

A follow up visit to the Clinic was made by patient 30 days post procedure. The patient presented with a full growth of hair at the donor site, no visible evidence of wounds and substantial and complete hair growth at the transplant site. Using other techniques for hair transplant wound healing would normally take 6 and 8 weeks; hair growth evidence appears at the donor site between 8 and 12 weeks and 6 months at the transplant site.

After careful investigation by clinic staff of both donor and transplant sites there was no scaring, where normally there is; no hair loss where normal rates range from 20-70%; and dramatic hair growth rates in a short period of time. A subsequent follow up was conducted 2 weeks later and hair samples were taken to measure strength and vitality. All samples tested as normal healthy hair.

We claim:

1. Apparatus for controlled administration of electromagnetic wave energy and electromagnetic field energy for treating biological cellular structure comprising:
a) a programmable source of electromagnetic wave energy emitting incoherent light of about 380 to 840 nanometer wave length range, said source of wave energy being positioned within a parabolic, faceted reflector for directing an electromagnetic wave energy beam in a homogeneous spot focused on the cellular structure being treated;
b) multiple, programmable sources of electromagnetic wave energy comprising coherent light of about 690 to 880 nanometer wavelength range, which sources can be separately controlled, said sources of coherent light being positioned within a faceted reflector for directing the electromagnetic wave energy beam in a homogeneous spot focused on the cellular structure being treated, which spot is essentially coextensive with the spot emanating from the incoherent light;
c) multiple, programmable sources of electromagnetic field energy, including a first set of coils comprising coated wire located between the sources of electromagnetic wave energy and the cellular structure being treated, each source of electromagnetic field energy capable of being separately controlled, and at least one of the first set of coils being wound from bifilar wire; and
d) a digital control module for electronically controlling at least one of the frequency, duration, phase, duty cycle, pulse or amplitude of electromagnetic wave energy and electromagnetic field energy emitted from the light sources and coils.

2. The apparatus of claim 1 wherein the biological cellular structure being treated is that of a human and the apparatus further comprises:
a) a platform supporting the human in a position adjacent the light sources and first set of coils; and
b) additional sources of electromagnetic field energy, including a second set of coils located in the platform at one or more locations adjacent portions of the human cellular structure being treated.

3. The apparatus of claim 1 wherein the source of incoherent light is a halogen lamp.

4. The apparatus of claim 1 wherein the interior of the parabolic, faceted reflector is coated with at least one metallic layer that selectively permits reflection and transmission of light waves in the visible to near infrared wavelength range creating a cold light with substantially no thermal effect nor ionizing radiation.

5. The apparatus of claim 4 wherein the coated metallic layer comprises one or more metal oxides selected from the group consisting of quartz and titanium.

6. The apparatus of claim 1 wherein the source of coherent light is one or more light emitting diodes.

7. The apparatus of claim 1 wherein the bifilar wire consists essentially of a stainless steel alloy.

8. The apparatus of claim 7 wherein the stainless steel alloy wire is coated with a polyamide material.

9. The apparatus of claim 2 wherein the platform comprises multiple adjustable segments supporting different anatomical portions of the treated human being.

10. The apparatus of claim 2 wherein one or more of the second set of coils can be selectively energized by the control module to control at least one of the frequency, duration, phase, duty cycle, pulse or amplitude, of the electromagnetic field energy emitted from the second set of coils.

11. The apparatus of claim 1 wherein the control module is digital and is capable of receiving and executing preprogrammed treatment regimens.

12. The apparatus of claim 1 wherein focused beams from the incoherent and coherent lights overlap and form a homogeneous spot about 7-10 inches in diameter at the point where they impinge on the biological cellular structure.

13. The apparatus of claim 1 wherein there is substantially no thermal effect on cellular structure from the incoherent and coherent light beams impinging on that structure.

14. The apparatus of claim 1 wherein there is substantially no ionizing radiation on cellular structure from the in coherent and coherent light beams impinging on that structure.

15. A system for the treatment of human cellular structure comprising:
a) a programmable source of electromagnetic wave energy emitting incoherent light of about 380 to 840 nanometer wave length range, said source of wave energy being positioned within a parabolic, faceted reflecting means for directing the electromagnetic wave energy in a beam focused on the cellular structure being treated;
b) multiple, programmable sources of electromagnetic wave energy comprising coherent light of about 690 to 880 nanometer wavelength range, which sources can be separately controlled, said sources of coherent light being positioned within a faceted reflector for directing the electromagnetic wave energy in a beam focused on the cellular structure being treated, which beam is essentially coextensive with the beam emanating from the incoherent light;
c) sources of electromagnetic field energy, including a first set of coils comprising coated wire located between the sources of electromagnetic wave energy and the cellular structure being treated, and each source of electromagnetic field energy capable of being separately controlled;
d) a platform supporting the human in a position adjacent the light sources and first set of coils;
e) additional sources of electromagnetic field energy, including a second set of coils located in the platform at one or more locations adjacent portions of the human cellular structure being treated; and
f) a digital control module for electronically controlling at least one of the frequency, duration, phase, duty cycle, pulse or amplitude of electromagnetic wave energy and electromagnetic field energy emitted from the light sources and coils.
